(19) United States
(12) Patent Application Publication
(10) Pub. No.: US 2006/0264439 A1
(43) Pub. Date: Nov. 23, 2006

(54) Inhibitors of polo-like kinase-1

(75) Inventors: David J. Bearss, Highland, UT (US); Hariprasad Vankayalapati, Draper, UT (US); Cory L. Grand, Salt Lake City, UT (US)

Correspondence Address: SEED INTELLECTUAL PROPERTY LAW GROUP PLLC, 701 FIFTH AVE, SUITE 6300, SEATTLE, WA, 98104-7092, US

(73) Assignee: SuperGen, Inc., Dublin, CA (US)

(21) Appl. No.: 11/436,115

(22) Filed: May 17, 2006

Related U.S. Application Data
(60) Provisional application No. 60/681,815, filed on May 17, 2005.

(51) Int. Cl. C07D 237/28; A61K 31/502

(52) U.S. Cl. 514/248; 544/235

(57) ABSTRACT

Protein kinase inhibitors are disclosed having utility in the treatment of protein kinase-mediated diseases and conditions, such as cancer. The compounds of this invention have the following structure: 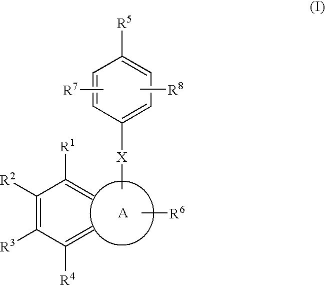
including steroisomers, prodrugs and pharmaceutically acceptable salts thereof, wherein ring moiety A, R₁, R₂, R₃, R₄, R₅, R₆, R₇, R₈ and X are as defined herein. Also disclosed are compositions comprising such compounds and methods of using same.

### BACKGROUND OF THE INVENTION

1. Field of the Invention

The present invention relates generally to compounds that inhibit kinase activity, and to compositions and methods related thereto.

2. Description of the Related Art

Polo-like kinase-1 (Plk-1) has emerged as an important new target for drug discovery and development. Plk-1 is a member of a family of polo-like kinase proteins which have been implicated in multiple pathways controlling cellular proliferation. This family was originally discovered in the fruit fly Drosophila melanogaster as Polo, a protein kinase which, when absent, leads to improper cellular division due to an inability to form a functional mitotic spindle apparatus (Sunkel, C. E. et al. (1988) J Cell Sci 89 (Pt 1), 25-38). Plk-1 plays a role at centrosomes where it regulates the separation and maturation of this structure. This is evidenced by the ability of anti-Plk-1 antibodies to disrupt chromosome separation (Lane, H. A., et al. (1996) J Cell Biol 135, 1701-1713). Other apparent roles for Plk-1 include control of mitotic entry and exit (Nigg, E. A. (1998) Curr Opin Cell Biol 10, 776-783), direction of Cyclin B1 to the nucleus at the start of mitosis (Toyoshima-Morimoto et al. (2001) Nature 410, 215-220), activation of the proliferation-inducing phosphatase cdc25 (Roshak, A. K., et al. (2000) Cell Signal 12, 405-411), and repression of cdc2-Cyclin B1, which signals progression out of M-phase (Nigg, E. A. (1998) Curr Opin Cell Biol 10, 776-783). Plk-1 protein is also targeted for degradation in response to DNA damage at the G2 phase, further establishing its importance in cell cycle progression (Kang, D., et al. (2002) J Cell Biol 156, 249-259).

High levels of Plk-1 have been associated with poor prognosis in a number of human tumor types, including breast (Wolf, G., et al. (2000) Pathol Res Pract 196, 753-759), colon (Macmillan, J. C., et al. (2001) Ann Surg Oncol 8, 729-740), endometrial (Takai, N., et al. (2001) Cancer Lett 169, 41-49), esophageal (Tokumitsu, Y., et al. (1999) Int J Oncol 15, 687-692) and oropharyngeal carcinomas (Knecht, R., et al. (2000) Int J Cancer 89, 535-536), non-small cell lung cancer (Wolf, G., et al. (1997) Oncogene 14, 543-549), melanoma (Strebhardt, K., et al. (2000) Jama 283, 479480) and squamous cell carcinoma (Knecht, R., et al. (1999) Cancer Res 59, 2794-2797). The oncogenic nature of Plk-1 has been further confirmed by its ability to cause transformation of NIH3T3 mouse fibroblasts and tumor formation in mice (Smith, M. R., et al. (1997) Biochem Biophys Res Commun 234, 397-405).

The inhibition of Plk-1 has significant antitumor effects in a number of different cancer types. For example, treatment of human prostate cancer cell lines with small interfering RNAs (siRNAs) directed against Plk-1 led to a decrease in cell viability, induction of an apoptotic response, mitotic arrest, cytokinetic failure and defects in centrosome integrity and maturation (Reagan-Shaw, S. R., et al. (2004) Proceedings of the AACR 45). This effect was not seen with normal human prostate epithelial cells. Similar effects in other cell types were observed, including breast, cervical, colorectal and lung carcinoma cell lines, while nontransformed cells from the same source were resistant to the effects of Plk-1 RNA silencing (Spankuch-Schmitt, B., et al. (2002) J Natl Cancer Inst 94, 1863-1877). Antisense oligonucleotide strategies directed against Plk-1 have also yielded clinically relevant results (e.g., cell cycle arrest, decreased proliferation and increased apoptosis) in pancreatic carcinoma. Promising results have also been observed in vivo, where treatment with anti-Plk-1 short hairpin RNA (shRNA) caused tumor shrinkage in human tumor xenograft mouse models (Spankuch-Schmitt, B., et al. (2003) Clinical Cancer Research 9, A243).

Finally, indirect methods of inhibiting Plk-1 have also been studied, such as disruption of the heat-shock protein Hsp90, which leads to an inability of centrosomes to nucleate microtubules and can be rescued with recombinant Plk-1 (de Carcer, G., et al. (2001) Embo J 20, 2878-2884) and single-dose ionizing radiation (which leads to a reduction in Plk-1 gene expression and G₂/M blockade) (Bratland, A., et al. (2003) Proceedings of the AACR 44, 2nd Ed). The above results taken together underscore the importance of Plk-1 as a target for the development of novel therapeutic compounds and methods.

A natural product inhibitor of polo-like kinase 1 has been described. Scytonemin, a pigment isolated from blue-green algae and investigated in relation to its anti-inflammatory properties, has recently been identified as an inhibitor of Plk-1 activity. This compound causes apoptosis in Jurkat T cells (Stevenson, C. S., et al. (2002) J Pharmacol Exp Ther 303, 858-866).

What remains needed, however, are additional and improved inhibitors of protein kinase activity, particularly compounds effective for modulating Plk-1 activity. The present invention fulfills these needs and offers other related advantages.

### BRIEF SUMMARY OF THE INVENTION

In one embodiment, the present invention provides compounds having the following structure (I): 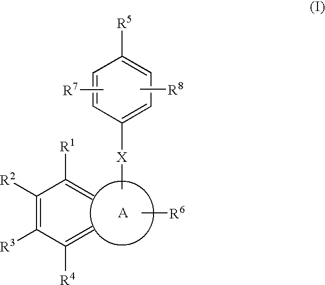
including steroisomers, prodrugs and pharmaceutically acceptable salts thereof, wherein A, R₁, R₂, R₃, R₄, R₅, R₆, R₇, R₈ and X are as defined herein.

Compounds of the present invention have utility over a broad range of therapeutic applications, and may be used to treat diseases, such as cancer and inflammatory conditions, that are mediated at least in part by protein kinase activity, particularly Plk-1 kinase activity.

Accordingly, in another aspect of the invention, the compounds described herein are formulated as pharmaceutically acceptable compositions for administration to a subject in need thereof.

Another aspect of the invention relates to a method for inhibiting protein kinase activity in a biological sample, which method comprises contacting the biological sample with a compound described herein, or a pharmaceutically acceptable composition comprising said compound, and thereby inhibiting protein kinase activity in the sample. In certain embodiments, the protein kinase is Plk-1 kinase.

Another aspect of this invention relates to a method of inhibiting protein kinase activity in a subject, which method comprises administering to a subject in need thereof a compound described herein or a pharmaceutically acceptable composition comprising said compound, and thereby inhibiting protein kinase activity in the subject. In certain embodiments, the protein kinase is Plk-1 kinase.

In another aspect, the invention provides methods for treating or preventing a protein kinase-mediated disease, such as cancer, which method comprises administering to a patient in need thereof a therapeutically effective amount of a compound described herein or a pharmaceutically acceptable compositions comprising said compound, and thereby treating the protein kinase-mediated disease. In certain embodiments, the protein kinase is Plk-1 kinase. In certain other embodiments, the protein kinase-mediated disease is a Plk-1-mediated disease such as, for example, pancreatic cancer, breast cancer, colon cancer, cervical cancer, endometrial cancer, esophageal cancer, oropharyngeal cancer, lung cancer (e.g., non-small cell lung cancer), skin cancer (e.g., melanoma) and squamous cell cancer.

These and other aspects of the invention will be apparent upon reference to the following detailed description and attached figures. Patent and other documents cited herein to more specifically set forth various aspects of this invention are hereby incorporated by reference in their entireties.

### DETAILED DESCRIPTION OF THE INVENTION

The present invention is generally directed to compounds useful as protein kinase inhibitors and to compositions and methods relating thereto. Such compounds have the following structure (I): 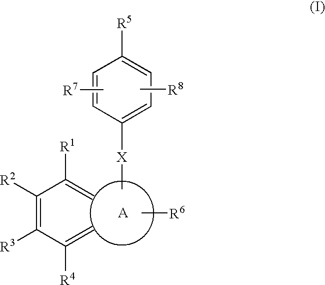
including steroisomers, prodrugs, ester amides and pharmaceutically acceptable salts thereof,

wherein:
ring moiety A represents a heterocycle;
R¹, R², R³ and R⁴ are the same or different and are independently hydrogen, alkyl, alkoxy, halo, haloalkyl, alkylthio, cyano, nitro, amine, alkylsulfanyl or —X¹R⁹;
R⁵ is hydrogen, hydroxyl, alkyl, alkoxy, halo, haloalkyl, alkylthio, cyano, nitro, amine, —C(═C), —NHC(═O)R¹⁰, —NHC(═S)NHR¹⁰, —NHS(═O)₂R¹⁰, —C(═O)R¹⁰, —C(═O)NHR¹⁰, —S(═O)₂NHC(═O)R¹⁰ or —S(═O)R¹⁰;
R⁶ is hydrogen, —C(═O)OH, —C(═O)NH₂, or —C(═O)R¹¹;
R⁷ and R⁸ are the same or different and are independently hydrogen, alkyl, alkoxy, halo, haloalkyl, alkylthio, cyano, nitro or amine;
R⁹ is a carbocycle, substituted carbocycle, heterocycle, substituted heterocycle, hydrogen, C₁₋₃ alkyl or C₁₋₃ alkoxy;
R¹⁰ is alkyl, substituted alkyl, carbocycle, substituted carbocycle, heterocycle or substituted heterocycle;
R¹¹ is a carbocycle, substituted carbocycle, heterocycle or substituted heterocycle;
X is —NH—, —N═C—, —O—, —S—, —S(═O)— or —S(═O)₂—; and
X¹ is a direct bond, —O—, —CH₂—, —OCO—, carbonyl, —S—, —S(═O)—, —S(═O)₂—, —C(═O)NH—, or —S(═O)₂NH—.

Unless otherwise stated the following terms used in the specification and claims have the meanings set forth below:

“Alkyl” refers to a saturated straight or branched hydrocarbon radical of one to six carbon atoms, preferably one to four carbon atoms, e.g., methyl, ethyl, propyl, 2-propyl, n-butyl, iso-butyl, tert-butyl, pentyl, hexyl, and the like, preferably methyl, ethyl, propyl, or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂-cyclohexenyl, and the like. Cyclic alkyls are also referred to herein as a “cycloalkyl.” Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like, preferably methylene, ethylene, or propylene.

“Cycloalkyl” refers to a saturated cyclic hydrocarbon radical of three to eight carbon atoms, e.g., cyclopropyl, cyclobutyl, cyclopentyl or cyclohexyl.

“Alkoxy” means a radical —ORₐ where Rₐ is an alkyl as defined above, e.g., methoxy, ethoxy, propoxy, butoxy and the like.

“Halo” means fluoro, chloro, bromo, or iodo.

“Haloalkyl” means alkyl substituted with one or more, preferably one, two or three, same or different halo atoms, e.g., —CH₂Cl, —CF₃, —CH₂CF₃, —CH₂CCl₃, and the like.

“Haloalkoxy” means a radical —OR_{b} where R_{b} is an haloalkyl as defined above, e.g., trifluoromethoxy, trichloroethoxy, 2,2-dichloropropoxy, and the like.

“Acyl” means a radical —C(O)R_{c} where R_{c} is hydrogen, alkyl, or haloalkyl as defined herein, e.g., formyl, acetyl, trifluoroacetyl, butanoyl, and the like.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. When substituted, the aryl group is substituted with one or more, more preferably one, two or three, even more preferably one or two substituents independently selected from the group consisting of alkyl, haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino or dialkylamino.

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be substituted or unsubstituted. When substituted, the heteroaryl group is substituted with one or more, more preferably one, two or three, even more preferably one or two substituents independently selected from the group consisting of alkyl, haloalkyl, halo, hydroxy, alkoxy, mercapto, alkylthio, cyano, acyl, nitro, haloalkyl, haloalkoxy, carboxy, alkoxycarbonyl, amino, alkylamino or dialkylamino.

“Carbocycle” refers to an aliphatic ring system having 3 to 14 ring atoms. The term “carbocycle”, whether saturated or partially unsaturated, also refers to rings that are optionally substituted. The term “carbocycle” also include aliphatic rings that are fused to one or more aromatic or nonaromatic rings, such as in a decahydronaphthyl or tetrahydronaphthyl, where the radical or point of attachment is on the aliphatic ring.

“Heterocycle” refers to a cyclic ring system, saturated or unsaturated, having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The heterocyclyl ring may be optionally substituted independently with one or more, preferably one, two, or three substituents selected from alkyl (wherein the alkyl may be optionally substituted with one or two substituents independently selected from carboxy or ester group), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, aralkyl, heteroaralkyl, saturated or unsaturated heterocycloamino, saturated or unsaturated heterocycloaminoalkyl, and —COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

“Optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocyclo group is not substituted with the alkyl group.

Lastly, the term “substituted” as used herein means any of the above groups (e.g., alkyl, akylene, cycloalkyl, alkoxy, haloalkyl, haloalkoxy, acyl, aryl, heteroaryl, carbocycle, heterocycle, etc.) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₑR_{f}, —NRₑC(═O)R_{f}, —NRₑC(═O)N RₑR_{f}, —NRₑC(═O)OR_{f} —NRₑSO₂R_{f}, —ORₑ, —C(═O)Rₑ —C(═O)ORₑ, —C(═O)NRₑR_{f}, —OC(═O)NRₑR_{f}, —SH, —SRₑ, —SORₑ, —S(═O)₂Rₑ, —OS(═O)₂Re, —S(═O)₂ORₑ, wherein Rₑ and R_{f} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

In a further aspect of structure (I) above, R¹, R², R³ and R⁴ are the same or different and are independently hydrogen, C₁-C₃ alkyl, C₁-C₃ alkoxy, halo, amine or nitro.

In a further aspect of structure (I) above, R⁵ is hydroxyl, —C(═C), —S(═O)₂NHC(═O)CH₃ or —C(═O)NHR¹⁰, where R¹⁰ is a carbocycle or substituted carbocycle.

In a further aspect of structure (I) above, R⁷ and R⁸ are the same or different and are independently hydrogen, C₁-C₃ alkyl, C₁-C₃ alkoxy, C₁-C₃ haloakyl, C₁-C₃ alkylthio, halo, cyano, nitro or amine.

In a further aspect of structure (I) above, ring moiety A has 6 ring atoms.

In a further aspect of structure (I) above, ring moiety A has 1 to 3 heteroatoms.

In a further aspect of structure (I) above, ring moiety A has 6 ring atoms and 1 to 3 heteroatoms.

In a further aspect of structure (I) above, ring moiety A has 6 ring atoms and 2 nitrogen heteroatoms.

In further aspects of structure (I) above, X is either —NH— or —N═C—.

In further aspects of structure (I) above, X is either —NH— or —N═C—, and the compounds have the following structures (IA-1), (IB-1) and (IC-1):

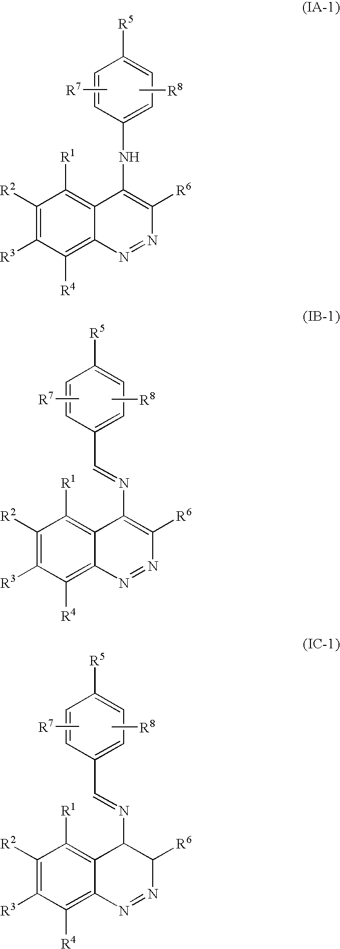

In a further aspect of structure (IA-1) above, R¹ and R⁴ are both hydrogen.

In a further aspect of structure (IA) above, R₆ is not hydrogen.

In a further aspect of structure (IA-1) above, R⁷ and R⁸ are both hydrogen.

In a further aspect of structure (IA-1) above, R² and R³ are methoxy.

In a further aspect of structure (IA-1) above, R² and R³ are methoxy and R¹, R⁴ and R⁶ are hydrogen.

In a further aspect of structure (IA-1) above, R² and R³ are methoxy, and R⁵ is —S(═O)₂NHC(═O)CH₃.

In a further aspect of structure (IA-1) above, R⁵ is —S(═O)₂NHC(═O)CH₃.

In a further aspect of structure (IA-1) above, R² and R³ are methoxy and R¹, R⁴ and R⁶ are hydrogen, and R⁵ is —S(═O)₂NHC(═O)CH₃.

In further aspects of structures (IA-1), (IB-1) and (IC-1) above, R¹, R⁴, R⁷ and R⁸ are all hydrogen, and the compounds have the structures (IA-2), (IB-2) and (IC-2):

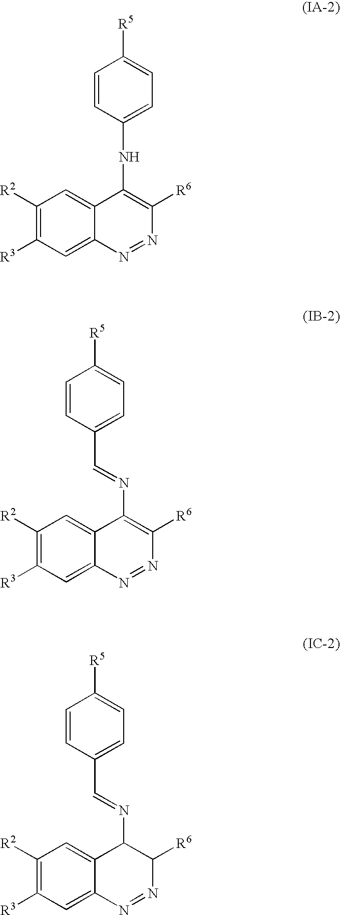

In a further aspect of structure (IA-2) above, R² and R³ are the same or different and are independently hydrogen, C₁-C₃ alkoxy, halo, nitro, amine, heterocycle or substituted heterocycle.

In a further aspect of structure (IA-2) above, R² and R³ are the same or different and are independently methoxy, —Cl, —F, —NH₂, —NO₂ or:

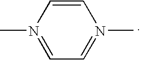

In a further aspect of structure (IA-2) above, R² is methoxy, —F, —NO₂ or —NH₂, and R³ is hydrogen, methoxy or —Cl.

In a further aspect of structure (IA-2) above, R⁵ is -hydroxyl, —C(═C) or —C(═O)NHR¹¹, where R¹¹ is a carbocycle or substituted carbocycle.

In a further aspect of structure (IA-2) above, R⁵ is —C(═O)NHR¹¹, where R¹¹ is a carbocycle or substituted carbocycle.

In a further aspect of structure (IA-2) above, R⁵ is —C(═O)NHR¹¹, R¹¹ is phenyl, and the compounds have the following structure (IA-3) below:

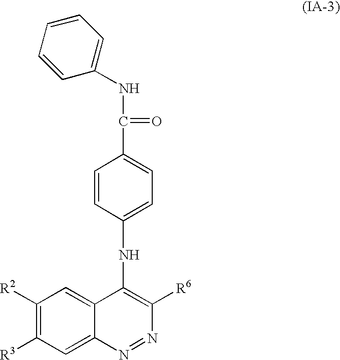

In a further aspect of structure (IA-2) above, R² and R³ are methoxy.

In a further aspect of structure (IA-2) above, R² and R³ are methoxy and R⁶ is hydrogen.

In a further aspect of structure (IA-2) above, R⁵ is —S(═O)₂NHC(═O)CH₃.

In a further aspect of structure (IA-2) above, R² and R³ are methoxy, and R⁵ is —S(═O)₂NHC(═O)CH₃.

In a further aspect of structure (IA-2) above, R² and R³ are methoxy, R⁶ is hydrogen, and R⁵ is —S(═O)₂NHC(═O)CH₃.

In a further aspect of structure (IA-3) above, R⁶ is —C(═O)NH₂ and the compounds have the following structure (IA-4):

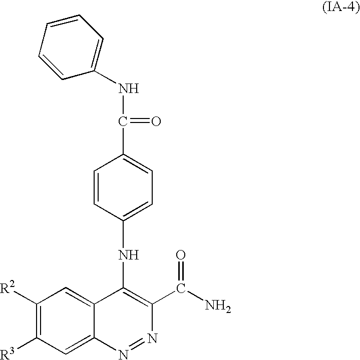

In a further aspect of (IA-4) above, R² and R³ are the same or different and are independently hydrogen, methoxy, —NH₂, —F, —Cl or —NO₂.

In a further aspect of (IA-4) above, R² and R³ are the same or different and are independently hydrogen, methoxy, —NH₂, —F, —Cl or —NO₂, and the compounds have the following structures (IA-5) to (IA-10) below:

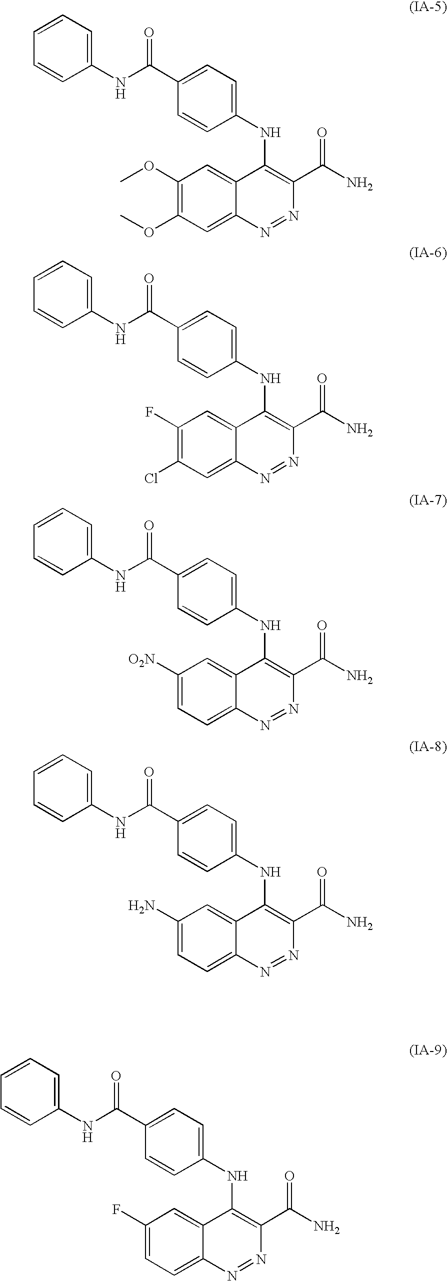

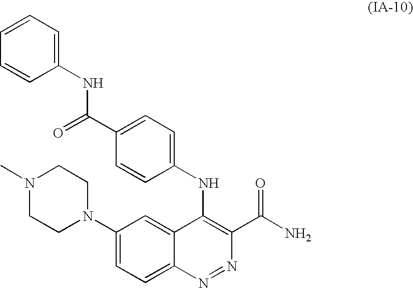

In a further aspect of (IA-3) above, R⁶ is —C(═O)R¹² where R¹² is a heterocycle.

In a further aspect of (IA-3) above, R⁶ is —C(═O)R¹², where R¹² is: 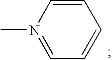
and the compounds have the following structures (IA-11) and (IA-12) below:

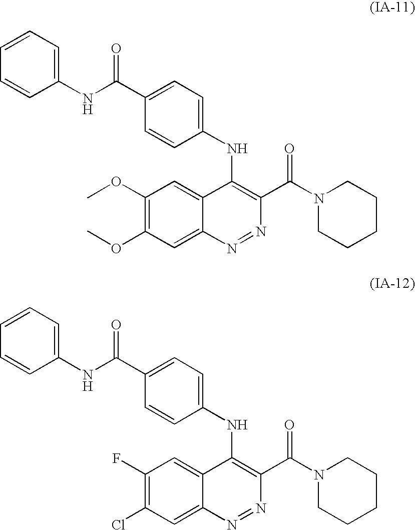

In a further aspect of structure (IB-2) above, R² and R³ are the same or different and are independently selected from hydrogen, C₁-C₃ alkoxy, halo, —NO₂, —NH₂, heterocycle or substituted heterocycle.

In a further aspect of structure (IB-2) above, R² and R³ are the same or different and are independently selected from halo.

In a further aspect of structure (IB-2) above, R⁵ is selected from nitro, hydrogen, hydroxyl or —C(═C).

In a further aspect of structure (IB-2) above, R² is —F and R³ is —Cl, and the compounds have the following structure (IB-3) below:

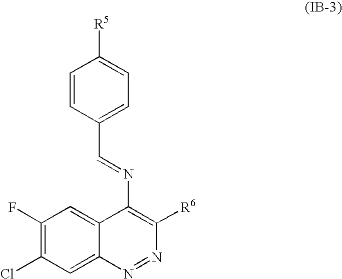

In a further aspect of structure (IB-3) above, R⁵ is selected from nitro, hydrogen, hydroxyl or —C(═C).

In a further aspect of structure (IB-3) above, R⁶ is selected from —C(═O)NH₂ or —C(═O)OH.

In a further aspect of structure (IB-3) above, R⁵ is selected from nitro, hydrogen, hydroxyl or —C(═C), R⁶ is selected from —C(═O)NH₂ or —C(═O)OH, and the compounds have the following structures (IB-4) to (IB-10) below:

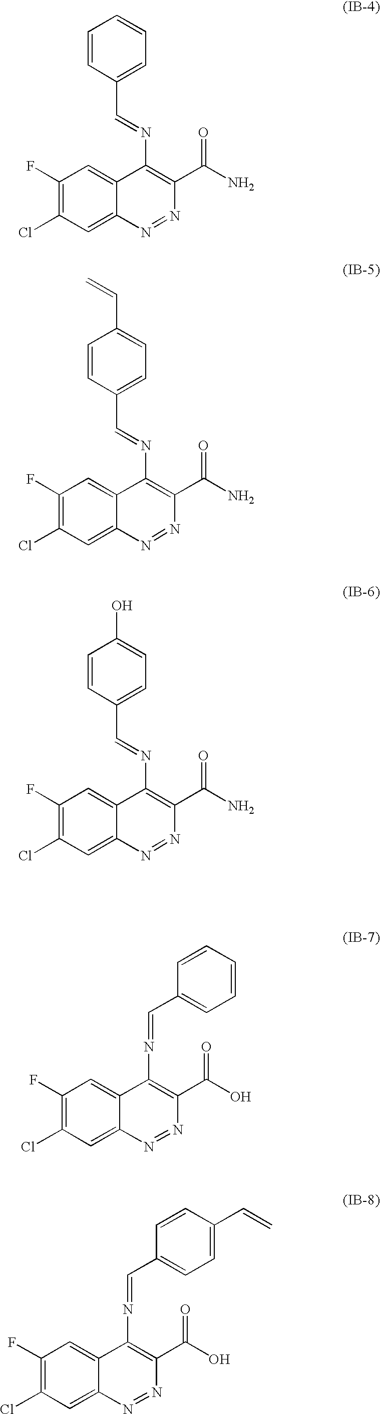

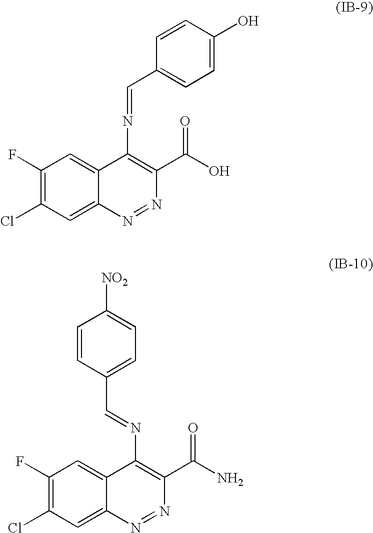

In a further aspect of structure (IC-2) above, R² and R³ are the same or different and are independently hydrogen, alkoxy, halo, —NO₂, —NH₂, heterocycle or substituted heterocycle.

In a further aspect of structure (IC-2) above, R² and R³ are the same or different and are independently selected from halo.

In a further aspect of structure (IC-2) above, R² is —F and R³ is —Cl, and the compounds have the following structure (IC-3) below:

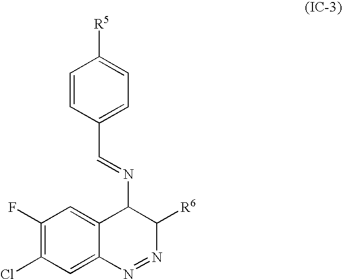

In a further aspect of structure (IC-3) above, R⁵ is selected from hydrogen, hydroxyl or —C(═C).

In a further aspect of structure (IC-3) above, R⁶ is selected from —C(═O)—NH2 or —C(═O)OH.

In a further aspect of structure (IC-3) above, R₅ is selected from hydroxyl or —C(═C), R⁶ is —C(═O)OH, and the compounds have the following structures (IC4) to (IC-5):

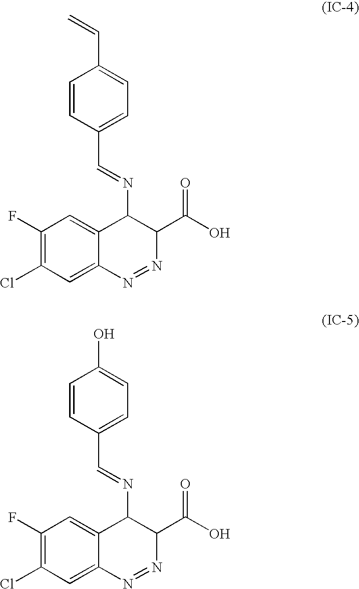

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog (Cahh, R., Ingold, C., and Prelog, V. Angew. Chem. 78:41347, 1966; Angew. Chem. Internat. Ed. Eng. 5:385-415, 511, 1966), or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Ch. 4 of ADVANCED ORGANIC CHEMISTRY, 4^{th} edition, March, J., John Wiley and Sons, New York City, 1992).

The compounds of the present invention may exhibit the phenomena of tautomerism and structural isomerism. For example, the compounds described herein may adopt an E or a Z configuration about a double bond connecting moieties or they may be a mixture of E and Z. This invention encompasses any tautomeric or structural isomeric form and mixtures thereof which possess the ability to modulate aurora-2 kinase activity and is not limited to, any one tautomeric or structural isomeric form.

It is contemplated that a compound of the present invention may be metabolized by enzymes in the body of an organism such as humans to generate a metabolite that can modulate the activity of the protein kinases. Such metabolites are also within the scope of the present invention.

In another embodiment, pharmaceutical compositions containing one or more compounds of this invention are disclosed, as well as methods for administering said compositions to an animal in need thereof, such as a warm-blooded mammal, preferably a human. For the purposes of administration, the compounds of the present invention may be formulated as pharmaceutical compositions. Pharmaceutical compositions of the present invention comprise a compound disclosed herein and a pharmaceutically acceptable carrier and/or diluent. The compound is present in the composition in an amount that is effective to treat a particular disorder of interest, and preferably with acceptable toxicity to the subkect. Typically, the pharmaceutical composition may include a compound of this invention in an amount ranging from 0.1 mg to 250 mg per dosage depending upon the route of administration, and more typically from 1 mg to 60 mg. One skilled in the art can readily determine appropriate concentrations and dosages using well known and established techniques in the art.

Pharmaceutically acceptable carrier and/or diluents are familiar to those skilled in the art. For compositions formulated as liquid solutions, acceptable carriers and/or diluents include saline and sterile water, for example, and may optionally include antioxidants, buffers, bacteriostats and other common additives. The compositions can also be formulated as pills, capsules, granules, or tablets that contain, in addition to a compound of this invention, dispersing and surface-active agents, binders, and lubricants. One skilled in this art may further formulate the compound in an appropriate manner, and in accordance with accepted practices, such as those disclosed in REMINGTON'S PHARMACEUTICAL SCIENCES, Gennaro, Ed., Mack Publishing Co., Easton, Pa. 1990.

These compounds and compositions of the present invention have utility over a broad range of therapeutic applications, and may be used to treat diseases, such as cancer and inflammatory conditions, that are mediated at least in part by protein kinase activity.

Accordingly, in another embodiment, the invention provides methods for treating or preventing a protein kinase-mediated disease, such as cancer, which method comprises administering to a patient in need of such a treatment a therapeutically effective amount of a compound described herein or a pharmaceutically acceptable compositions comprising said compound.

Another aspect of the invention relates to inhibiting protein kinase activity in a biological sample, which method comprises contacting the biological sample with a compound described herein, or a pharmaceutically acceptable composition comprising said compound.

Another aspect of this invention relates to a method of inhibiting protein kinase activity in a patient, which method comprises administering to the patient a compound described herein or a pharmaceutically acceptable composition comprising said compound.

The compounds and compositions of the invention may be applied as a sole therapy or may involve, in addition to one or more compounds of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat cancer. In medical oncology the other component(s) of such conjoint treatment may include surgery, radiotherapy and/or chemotherapy.

Combination chemotherapy can include, in addition to one or more compounds of the present invention, essentially any other known category of therapeutic agent. For example, the combination chemotherapy may comprise:

(i) cytostatic agents and combinations thereof, such as antioestrogens, for example tamoxifen, toremifene, raloxifene, droloxifene and/or iodoxyfene; progestogens such as megestrol acetate; aromatase inhibitors such as anastrozole, letrazole, vorazole and/or exemestane; antiprogestogens, antiandrogens such as flutamide, nilutamide, bicalutamide and/or cyproterone acetate; LHRH agonists and antagonists such as goserelin acetate and/or luprolide; inhibitors of testosterone 5 α-dihydroreductase such as finasteride; anti-invasion agents such as the metalloproteinase inhibitor marimastat and/or an inhibitor of urokinase plasminogen activator receptor function; inhibitors of growth factor function; antibodies specific for growth factors and/or growth factor receptors; tyrosine kinase inhibitors; serine/threonine kinase inhibitors, etc., and/or:

(ii) antiproliferative/antineoplastic drugs and combinations thereof, as used in medical oncology, including antimetabolites, for example antifolates such as methotrexate; fluoropyrimidines such as 5-fluorouracil; antitumor antibiotics such as anthracyclines like doxorubicin, daunomycin, epirubicin and idarubicin, mitomycin-C, dactinomycin, mithramycin, etc.; platinum derivatives such as cisplatin and/or carboplatin; alkylating agents such as nitrogen mustard, melphalan, chlorambucil, busulphan, cyclophosphamide, ifosfamide, nitrosoureas, and/or thiotepa; antimitotic agents such as vinca alkaloids like vincrisitine and taxoids like taxol and/or taxotere; topoisomerase inhibitors such as epipodophyllotoxins like etoposide and/or teniposide, amsacrine, topotecan, etc.).

The compounds of this invention can made by one skilled in this field according well known and established principles and techniques of synthetic organic chemistry. General procedures for the synthesis of cinnoline-based compounds are known (e.g., Nargund, L. V. G., Manohara, Y. N. and Hariprasad, V. (1994) Indian J. Chem 33B, 1001-1003; Laduree, D., Florentin, D. and Robba, M. (1980) J. Heterocycl Chem. 17, 1189-1193; WO2004/016615; U.S. Pat. No. 4,591,589; WO97/34876; Menon, G. and Purushothaman. (1994) Indian J. Chem. Soc., 70, 533-536; Kiselyov, A. and Dominguez, C. (1999) Tetrahedron, Lett., 40, 5111-5114, the contents of which are incorporated herein by reference). For purposes of illustration, and not by way of limitation, the compounds of the present invention can be made as provided herein, as well as by the more detailed procedures set forth in the Examples.

The invention can be further understood upon consideration of the following non-limiting Examples.

### EXAMPLES

### Example 1

### Molecular Modeling of Plk-1 Active Site

This example describes structural analysis of the kinase domain of Plk-1 using 3-dimensional modeling and structure-based design of ligands that competitively bind to the ATP binding pocket of Plk-1. PLK-1_HUMAN, accession P53350, was retrieved from the Swissprot database and the full length sequence (1-601 aa) of Plk-1 was used for predicting 3-dimensional protein structure based on a homologous sequence search of the EMBL database. The EMBL search with the sequence of the serine/threonine kinase domain of human Plk-1 retrieved high sequence similarity to the polo-box domain of Plk-1 (1Q4K), cAMP dependent protein kinase (1CDK), Aurora-2 kinase (1MUO, 1MQ4, 1OL5), and cAMP-dependent kinase (1APM), of which the three-dimensional structures have been determined. Sequences were retrieved from the Protein Data Bank (PDB) and an alignment of the sequences was performed. The polo-box domain of Plk-1 was not used in the sequence alignment since the kinase domain of Plk-1 is in the N-terminal region of the sequence. Secondary structural information was used to adjust the sequence alignment using Clustal W, since this can effect the accuracy of a modeled structure. The final sequence alignment was checked using the structure-structure alignment within the homology module of MOE (Molecular Operating Environment) software (Chemical Computing Group, Montreal, Quebec, Canada). Aurora-2 kinase crystal structures 1 MUO and 1MQ4 were found to have 33% sequence identity and were used as template structures for subsequent homology modeling.

Homology modeling was performed using the MOE software. Amino acid substitutions were built using a side chain rotamer library. Deletions and insertions were constructed by means of a loop database search. The initial model was refined in a stepwise manner by energy minimization using the Amber force field. First, the loops were refined with 1000 steps of minimization with a fixed and a free backbone, respectively. Then, all side chains with a constrained backbone were minimized for 1000 steps, followed by another 2000 steps of minimization for the whole model within the homology modeling function of MOE. The crude model from MOE was minimized with a few thousand cycles of minimization using the ABNR (adopted-basis Newton-Raphson) method. The best-scoring model was minimized to a root-mean-squared gradient of 1 kcal/(mol·Å).

The positions of the Mg2⁺ ion were modeled to those found in the crystal structure of 1CDK, 1MQ4 using Schrodinger software (Schrodinger, Inc., Portland, Oreg.). Further energy minimization was performed using Schrodinger where the entire protein was relaxed using the AMBER force field as implemented in Schrodinger running on Linux 9.0.

### Example 2

### Glide Docking and Virtual Screening of PLK-1 Active Site

Both crystal structure complexes (1CDK, 1MUO, 1MQ4) and the model of Plk-1 protein preparations were performed prior to docking using Glide analysis (Schrodinger, Inc. Portland, Oreg.). Compounds AMP-PNP, ATP and ADP were docked to both models. The results showed that all three ligands given highest scores and lowest energies in binding poses corresponded to those observed experimentally for similar ligands. As an internal test, the highest-scoring (Glidescore) poses of 1 of ADP docked to the 1MQ4 structure, which also had the lowest interaction energies, had rms deviations from the crystal structure of around 0.2-0.5 Å or less. In addition, the docked position of ADP in 1 MQ4 closely resembles the binding mode that has been depicted for the experimental complex, giving confidence in Glide's ability to also predict the binding mode. Similarly all three structures were docked into the ATP binding site of the homology structure of Plk-1 to confirm the mode of binding. The active site residues were defined for initial Glide Grids and docking protocol.

Based on the binding modes of these purine bases, docking of several known ligands of serine/threonine kinase inhibitors, as well as database structures, were screened virtually and this analysis identified novel pharmacophore cinnoline-containing structural moieties as inhibitors of Plk-1 activity. Illustrative examples of certain cinnoline-based compounds according to the present invention are provided below in Table 1.

TABLE 1

| NO | STRUCTURE |
|---|---|
| 1 | 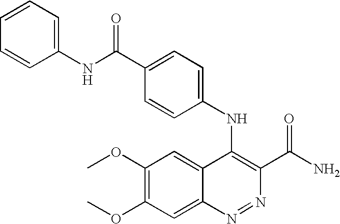 |
| 2 | 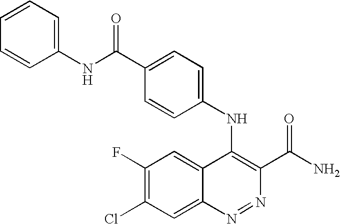 |
| 3 | 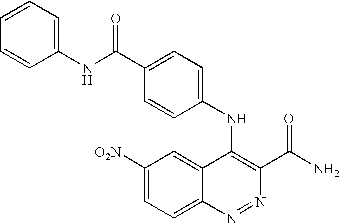 |
| 4 | 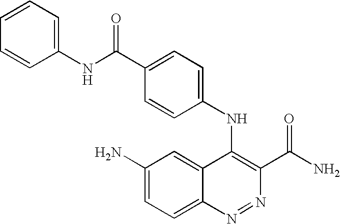 |
| 5 | 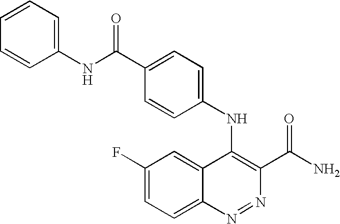 |
| 6 | 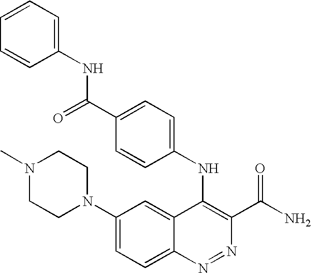 |
| 7 | 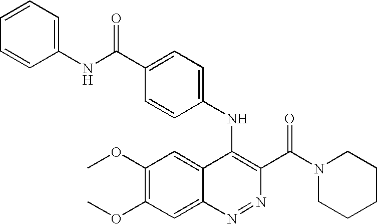 |
| 8 | 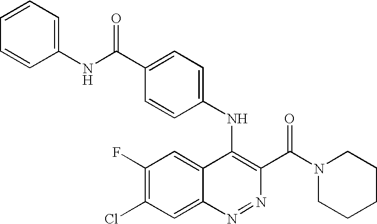 |
| 9 | 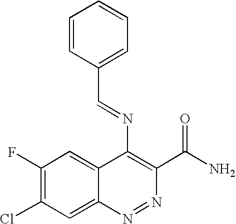 |
| 10 | 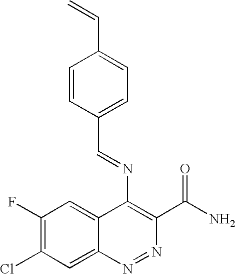 |
| 11 | 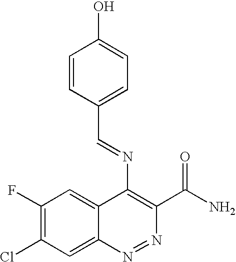 |
| 12 | 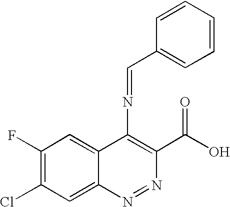 |
| 13 | 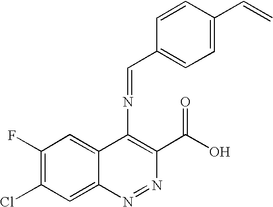 |
| 14 | 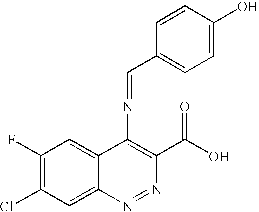 |
| 15 | 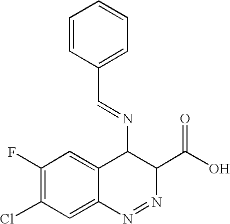 |
| 16 | 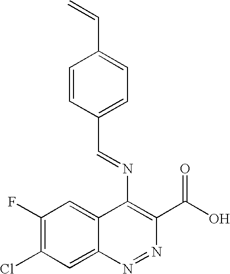 |
| 17 | 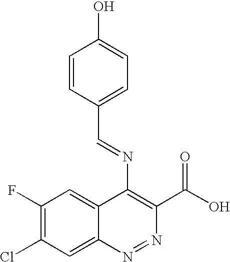 |
| 18 | 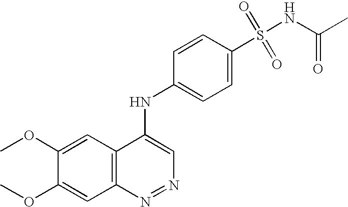 |

### Example 3

### Synthesis of PLK-1 Inhibitors

Synthesis of certain compounds of the invention was carried out using an approach depicted illustratively in Scheme 1 below: 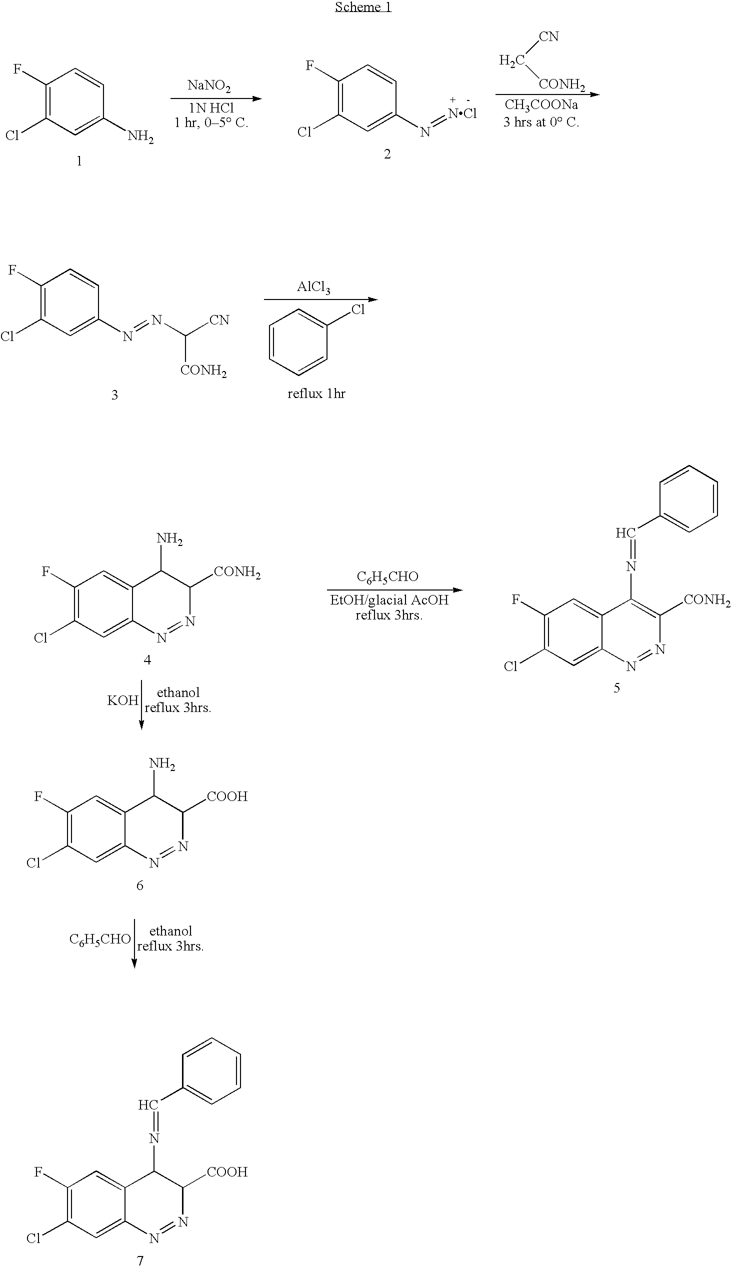
Chemical Synthesis of PLK-1 Inhibitors

¹H NMR spectra were recorded on a Varian 400 spectrometer, using the solvent as internal standard. Chemical shifts are expressed in ppm (δ). Proton magnetic resonance chemical shift values were measure in deuterated CDCl3 or DMSOd6 unless otherwise stated. ESI mass spectra (MS) were obtained on a VG-Quattro II and PE-SEIEX (API) mass spectrometer. Thin-layer chromatography was performed on Merck Kieselgel silica 60 plates coated with 250 μm layer with fluorescent indicator. Components were visualized by UV light (λ=254 nm) and or by iodine vapor. Flash column chromatographic separations were carried out on 70-230 mesh 60 Å silica gel and on CombiFlash companion (Teledyne ISCO) using RediSep flash columns. All the solvents used were best grade anhydrous obtained from Aldrich. Analytical HPLC was performed on a Waters Breeze system using the following and quoted as retention time (RT) in minutes. The column used was symmetry C18 5 μm, 4.6×150 mm column (WAT045905). All experiments dealing with moisture-sensitive compounds were conducted under dry nitrogen or argon. Starting materials, unless otherwise specified, were commercially available (Aldrich, Fluka, Lancaster and TCI) and of the best grade and were used without further purification. Organic solutions, where applicable, were dried over anhydrous Na₂SO₄ and evaporated using a Yamamto RE500 rotary evaporator at 15-20 mmHg.

### A. Synthesis of Substituted phenylhydrazino(cyano)acetamide (3)

To a suspension of 3-chloro-4-fluoroaniline (10 g) in 1N HCl (200 mL) was added NaNO₂ (4.7g dissolved in 40 ml H₂O) and the mixture stirred for 1 hr at 0-5° C. and the diazonium salt (compound 2) thus obtained was added to a well stirred mixture of cyanoacetamide (1.56 g), ethanol (30 mL) and water (100 mL) at 0° C. Sodium acetate was added in small portions during the reaction to keep the mixture alkaline and the mixture was stirred for 3 hrs at 0° C. The precipitated diazene compound was collected, washed thoroughly with water, air-dried and crystallized from ethanol to give compound 3.

### B. Synthesis of 4-amino-7-chloro-6-fluoro-3,4-dihydrocinnoline-3-carboxamide 4)

A mixture of compound 3, chlorobenzene and anhydrous AlCl3 was stirred for 1 hr. reflux under anhydrous conditions, cooled and poured onto ice water, and HCl was added while stirring. The precipitated product thus obtained was washed with pet.ether and made alkaline with ammonia. The pure base was filtered and washed with DMF and the obtained product 4 was dried.

### C. Synthesis of substituted 4N(arylidine)-7-chloro-6-fluorocinnoline-3-carboxamide (5)

4-amino-7-chloro-6-fluorocinnoline-3-carboxamide in ethanol and substituted aryl aldehyde was refluxed for 3 hrs in the presence of ACOH. The resulting solution was cooled and poured on to ice and the solid thus separated was filtered washed with water, dried and crystallized from DMF to give compound 5.

### D. Synthesis of 4.4-amino-7-chloro-6-fluorocinnoline-3-carboxylic acid (6)

A mixture of compound 4 and KOH was refluxed in ethanol for 3 hrs. After the reaction it was cooled and poured into water and neutralized with AcOH. The obtained product was filtered and crystallized from ethanol to give compound 6.

### E. Synthesis of substituted 4N(arylidine)-7-chloro-6-fluorocinnoline-3-carboxylic acid (7)

4-amino-7-chloro-6-fluorocinnoline-3-carboxylic acid 6 in ethanol and substituted aryl aldehyde was refluxed for 3 hrs in the presence of ACOH. The resulting solution was cooled and poured on to ice and the solid thus separated was filtered washed with water, dried and crystallized from DMF to give compound 7.

### F. Synthesis of (E)-4-(4-nitrobenzylideneamino)-7-chloro-6-fluorocinnoline-3-carboxamide (Compound IB-10)

A mixture of compound 4 (Scheme1) and 4-nitrobenzaldehyde in ethanol was refluxed for 3 hours. The clear solution was cooled and poured in ice water. The solid thus separated was filtered, washed with water, dried and crystallized from DMF to give compound IB-10.

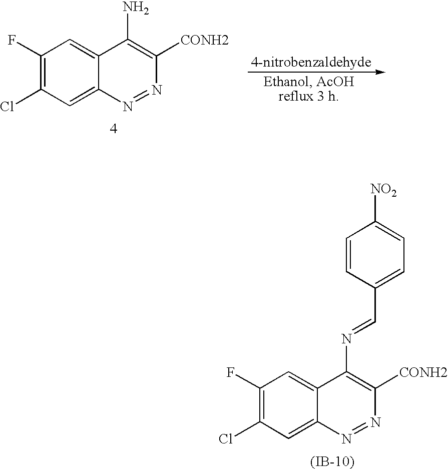

### G. Synthesis of N-Acetyl-4-(6,7-dimethoxy-cinnolin-4-ylamino)-benzenesulfonamide (Table 1, Compound 18)

N-Acetyl-4-(6,7-dimethoxy-cinnolin-4-ylamino)-benzenesulfonamide was also synthesized using a related approach. Briefly, a solution of DMA (dimethylacetamide) 10 mL was added to the compound sulfacetamide 47.68 mg (0.223 mmol), followed by the addition of 4-chloro-6,7-dimethoxycinnoline (50 mg, 0.223 mmol). The solution was stirred at reflux temperature for 10 hrs. After the completion of reaction, the solution was cooled, the solvents were evaporated and water was added. The obtained crude product was purified by CombiFlash Companion using a DCM and 1% MeOH solvent system (4 g normal phase RediSep Flash column with run time 40 min at flow 18 mL/min) and yielded 19 mg (21.21%) of white solid.

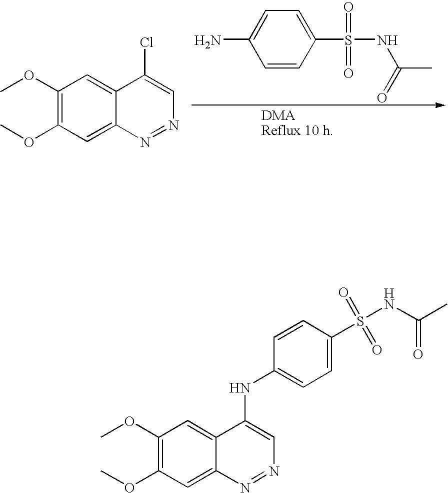

### Example 4

### Plk-1 Kinase Activity Assays

A. Plk-1 Kinase Inhibition Assay

One illustrative manner in which Plk-1 kinase activity can be determined is by quantifying the amount of ATP remaining in solution following the kinase reaction by measuring the relative light units (RLU) produced by luciferase using a luminometer. Percent activity is determined for individual compounds by comparing luminometer readings of compound-treated reactions to controls containing no compound (RLU_{No Inhib}) and no Plk-1 enzyme (RLU_{No Kinase}) in the following equation:

$$Percent Inhibition = \frac{{RLU}_{No Kinase} - {RLU}_{drug}}{{RLU}_{No Kinase} - {RLU}_{No Inhib}} \times 100$$

In a 50 μl reaction, 20 ng of recombinant polo-like kinase (e.g., Cell Signaling Technologies, Beverly, Mass.) is incubated at 30° C. for two hours with shaking (360 rpm) with 62.5 μM Kemptide (Calbiochem, San Diego, Calif.), 3 μM ATP (Invitrogen, Carlsbad, Calif.) and kinase reaction buffer (40 mM Tris-HCl, 20 mM MgCl₂ and 0.1 μg/μl bovine serum albumin). The value of 3 μM ATP was determined to be the Km (concentration at which the enzyme is working at 50% maximum velocity) for the amount of enzyme used in this assay. This reaction is carried out in the presence of one or more compounds of the present invention, diluted to desired concentrations in, for example, DMSO. After incubation, 50 μl of Kinase-Glo® (Promega, Inc., Madison, Wis.) solution is added to each reaction mixture and allowed to equilibrate for 10 minutes at room temperature. Kinase-Glo solution contains luciferase enzyme and luciferin, which react with ATP to produce light. Kinase activity is determined by quantifying the amount of ATP remaining in solution following the kinase reaction by measuring the relative light units (RLU) produced by luciferase using a luminometer (Thermo Electron Corporation, Vantaa, Finland).

B. Cell-Based Plk-1 Kinase Inhibitor Assays:

Cell culture-based assays can be used to evaluate the ability of compounds of the invention to inhibit one or more cellular activities, such as cancer cell growth and/or survival. Numerous cancer cell lines can be obtained from the American Type Culture Collection (ATCC) and other sources. Briefly, cells are seeded into 96-well, tissue-culture treated, opaque white plates (Thermo Electron, Vantaa, Finland), at between 5000 and 10000 cells per well, depending on the speed of cell proliferation, in 100 μl of appropriate growth medium (determined by the ATCC). Cells are then exposed to the appropriate concentration of drug or an equal amount of DMSO (drug diluent) and allowed to grow in its presence for 48 hours. Following this, 100 μl of Cell-Titer-Glo reagent (Promega, Inc., Madison, Wis.) is added to each well. Plates are then shaken for 2 minutes at room temperature to allow for cell lysis and incubated for 10 minutes to stabilize the luminescent signal. Similar to the Kinase-Glo assay reagent, this reagent contains both luciferase enzyme and its substrate luciferin. Luciferase, activated by ATP in the cell lysate, catalyzes the conversion of luciferin to oxyluciferin, a reaction which produces light. The amount of light produced is proportionate to the amount of ATP in the cell lysate, which is itself proportional to cell number and gives an index of cellular proliferation.

### Example 5

### Plk-1 Inhibitory Activity of Illustrative Compound (IB-10)

An ELISA-based Polo-like Kinase-1 activity assay (Cyclex, Nagano Japan) was used to confirm the Plk-1 inhibitory activity of the illustrative compound of the invention having the structure shown below.

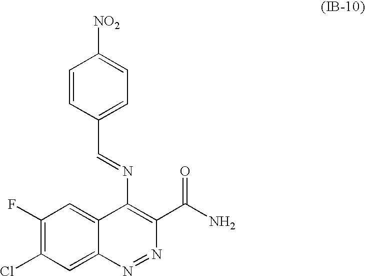

Briefly, 10 mU of purified Plk-1 (Cyclex) was incubated with the compound of interest in triplicate wells of a substrate-coated 96-well plate, along with 50 uM ATP and kinase buffer, for 30 minutes at 30° C. Wells were washed and probed with the anti-phospho-threonine antibody, incubated for 30 minutes at room temperature, then washed and probed again with an HRP-Anti Rabbit IgG. HRP substrate was added and the extent of phosphorylation was measured using a dual-wavelength spectrophotometer. At a concentration of 10 uM, compound (IB-10) inhibited Plk-1 activity in this assay by greater than 60%.

### Example 6

### Plk-1 Inhibitory Activity of Illustrative Compound N-Acetyl-4-(6,7-dimethoxy-cinnolin-4-ylamino)-benzenesulfonamide

An ELISA-based Polo-like Kinase-1 activity assay (Cyclex, Nagano Japan) was used to confirm the Plk-1 inhibitory activity of the illustrative compound of the invention having the structure shown below (Compound 18, Table 1).

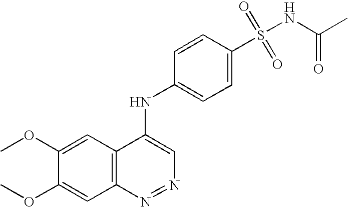

Briefly, 10 mU of purified Plk-1 (Cyclex) was incubated with the compound in triplicate wells of a substrate-coated 96-well plate, along with 50 uM ATP and kinase buffer, for 30 minutes at 30° C. Wells were washed and probed with the anti-phospho-threonine antibody, incubated for 30 minutes at room temperature, then washed and probed again with an HRP-Anti Rabbit IgG. HRP substrate was added and the extent of phosphorylation was measured using a dual-wavelength spectrophotometer. Using this approach, it was determined that at a concentration of 10 uM the compound reduced Plk-1 activity to 88% of untreated controls.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A compound having the following structure (I): 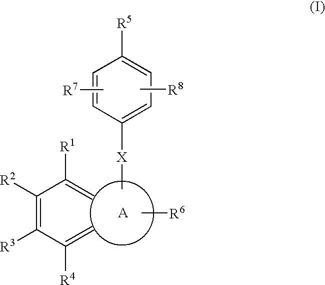
or a steroisomer, prodrug or pharmaceutically acceptable salt thereof,
wherein:
ring moiety A represents a heterocycle;
R¹, R², R³ and R⁴ are the same or different and are independently hydrogen, alkyl, alkoxy, halo, haloalkyl, alkylthio, cyano, nitro, amine, alkylsulfanyl or —X¹R⁹;
R⁵ is hydrogen, hydroxyl, alkyl, alkoxy, halo, haloalkyl, alkylthio, cyano, nitro, amine, —C(═C), —NHC(═O)R¹⁰, —NHC(═S)NHR¹⁰, —NHS(═O)₂R¹⁰, —C(═O)R¹⁰, —C(═O)NHR¹⁰, —S(═O)₂NHC(═O)R¹⁰ or —S(═O)R¹⁰;
R⁶ is hydrogen, —C(═O)OH, —C(═O)NH₂, or —C(═O)R¹¹;
R⁷ and R⁸ are the same or different and are independently hydrogen, alkyl, alkoxy, halo, haloalkyl, alkylthio, cyano, nitro or amine;
R⁹ is hydrogen, carbocycle, substituted carbocycle, heterocycle, substituted heterocycle, hydrogen, C₁₋₃ alkyl or C₁₋₃ alkoxy;
R¹⁰ is alkyl, substituted alkyl, carbocycle, substituted carbocycle, heterocycle or substituted heterocycle;
R¹¹ is a carbocycle, substituted carbocycle, heterocycle or substituted heterocycle;
X is —NH—, —N═C—, —O—, —S—, —S(═O)— or —S(═O)₂—; and
X¹ is a direct bond, —O—, —CH₂—, —OCO—, carbonyl, —S—, —S(═O)—, —S(═O)₂—, —C(═O)N—, or —S(═O)₂N—.

2. The compound of claim 1, where R¹, R², R³ and R⁴ are the same or different and are independently hydrogen, C₁-C₃ alkyl, C₁-C₃ alkoxy, halo, amine or nitro.

3. The compound of claim 1, where R⁵ is —C(═O)NHR¹⁰, where R¹⁰ is a carbocycle or substituted carbocycle.

4. The compound of claim 1, where R₆ is not hydrogen.

5. The compound of claim 1, where R⁷ and R⁸ are the same or different and are independently hydrogen, C₁-C₃ alkyl, C₁-C₃ alkoxy, C₁-C₃ haloakyl, C₁-C₃ alkylthio, halo, cyano, nitro or amine.

6. The compound of claim 1, where ring moiety A is a 6 membered heterocycle.

7. The compound of claim 1, where ring moiety A is a heterocycle having 1-3 nitrogen atoms.

8. The compound of claim 1, where ring moiety A is a 6 member heterocycle having 1-3 nitrogen atoms,

9. The compound of claim 1, where ring moiety A is a 6 member heterocycle having 2 nitrogen atoms.

10. The compound of claim 1, where X is —NH— or —N═C—.

11. The compound of claim 1, where X is —NH—, and the compound has the following structure (IA-1):

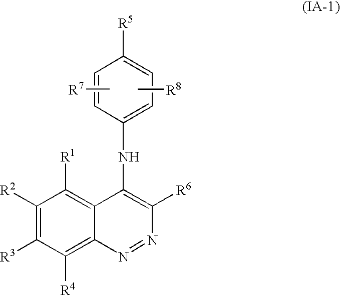

12. The compound of claim 1, where X is —N═C—, and the compound has the following structure (IB-1):

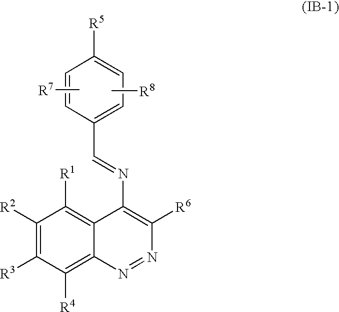

13. The compound of claim 1, where X is —N═C—, and the compound has the following structure (IC-1):

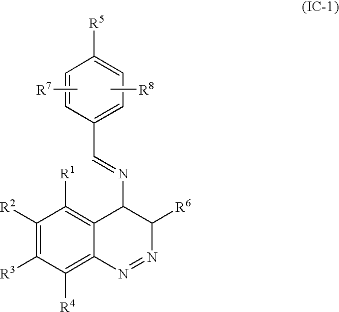

14. The compound of claim 11, where R¹ and R⁴ are both hydrogen.

15. The compound of claim 11, where R⁷ and R⁸ are both hydrogen.

16. The compound of claim 11, where R¹, R⁴, R⁷ and R⁸ are all hydrogen, and the compound has the following structure (IA-2):

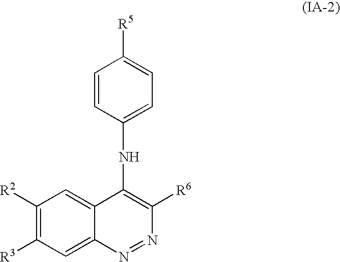

17. The compound of claim 12, where R¹, R⁴, R⁷ and R⁸ are all hydrogen, and the compound has the following structure (IB-2):

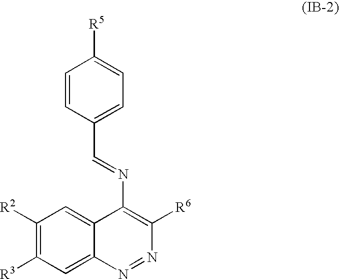

18. The compound of claim 13), where R¹, R⁴, R⁷ and R⁸ are all hydrogen, and the compound has the following structure (IC-2):

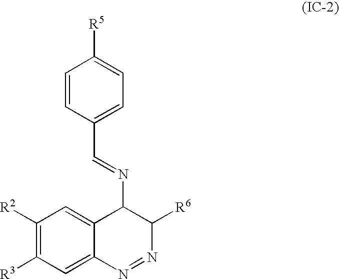

19. The compound of claim 16, where R² and R³ are the same or different and are independently hydrogen, C₁-C₃ alkoxy, halo, nitro, amine, heterocycle or substituted heterocycle.

20. The compound of claim 16, where R² and R³ are the same or different and are independently methoxy, —Cl, —F, —NH₂, —NO₂ or:

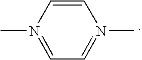

21. The compound of claim 16, where R² is methoxy, —F, —NO₂ or —NH₂, and R³ is hydrogen, methoxy or —Cl.

22. The compound of claim 16, where R⁵ is -hydroxyl, —C(═C), —S(═O)₂NHC(═O)CH₃ or —C(═O)NHR¹¹, where R¹¹ is a carbocycle or substituted carbocycle.

23. The compound of claim 16, where R⁵ is —C(═O)NHR¹¹, where R¹¹ is a carbocycle or substituted carbocycle.

24. The compound of claim 16, where R⁵ is —C(═O)NHR¹¹, R¹¹ is phenyl, and the compound has the following structure (IA-3):

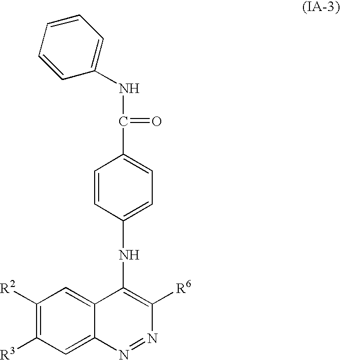

25. The compound of claims 16, where R² and R³ are methoxy and R⁶ is hydrogen.

26. The compound of claim 16, where R⁵ is —S(═O)₂NHC(═O)CH₃.

27. The compound of claim 16, where R² and R³ are methoxy, and R⁵ is —S(═O)₂NHC(═O)CH₃.

28. The compound of claim 16, where R² and R³ are methoxy, R⁶ is hydrogen, and R⁵ is —S(═O)₂NHC(═O)CH₃.

29. The compound of claim 11, where R⁶ is —C(═O)NH₂ and the compound has the following structure (IA-4):

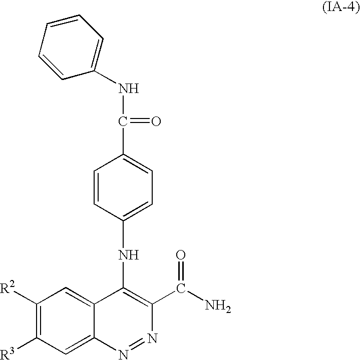

30. The compound of claim 29, where R₂ and R₃ are the same or different and are independently hydrogen, alkoxy, halo, alkyl, —NH₂ or —NO₂.

31. The compound of claim 29, where R₂ and R₃ are the same or different and are independently hydrogen, methoxy, —NH2, —F, —Cl or —NO₂.

32. The compound of claim 29, where the compound has a structure selected from the group consisting of structures (IA-5) to (IA-10) below:

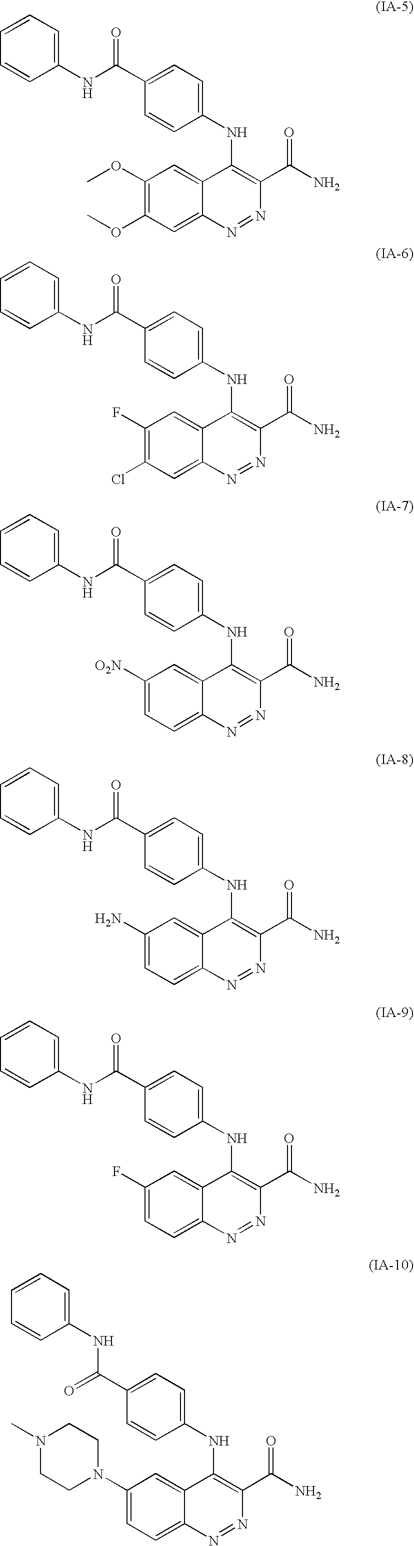

33. The compound of claim 24, where R⁶ is —C(═O)R¹² where R¹² is a heterocycle.

34. The compound of claim 24, where R⁶ is —C(═O)R¹², where R¹² is:

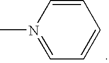

35. The compound of claim 24, where the compound has a structure selected from the group consisting of structures (IA-11) and (IA-12) below:

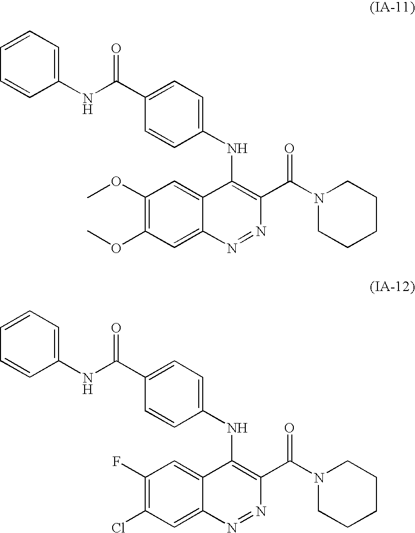

36. The compound of claim 17, where R² and R³ are the same or different and are independently selected from hydrogen, alkoxy, halo, —NO₂, —NH₂, heterocycle or substituted heterocycle.

37. The compound of claim 17, where R² and R³ are the same or different and are independently selected from halo.

38. The compound of claim 17, where R² is F and R³ is Cl and the compound has the following structure (IB-3) below:

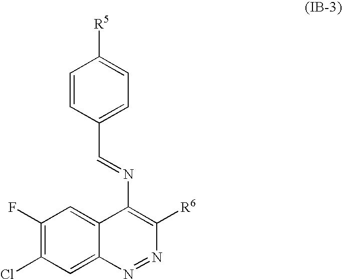

39. The compound of claim 38, where R⁵ is selected from nitro, hydrogen, hydroxyl or —C(═C).

40. The compound of claim 38, where R⁶ is selected from —C(═O)—NH₂ or —C(═O)OH.

41. The compound of claim 38, where R⁵ is selected from hydrogen, hydroxyl or —C(═C) and R⁶ is selected from —C(═O)—NH₂ or —C(═O)OH.

42. The compound of claim 38, where the compound has a structure selected from the group consisting of structures (IB-4) to (IB-10) below:

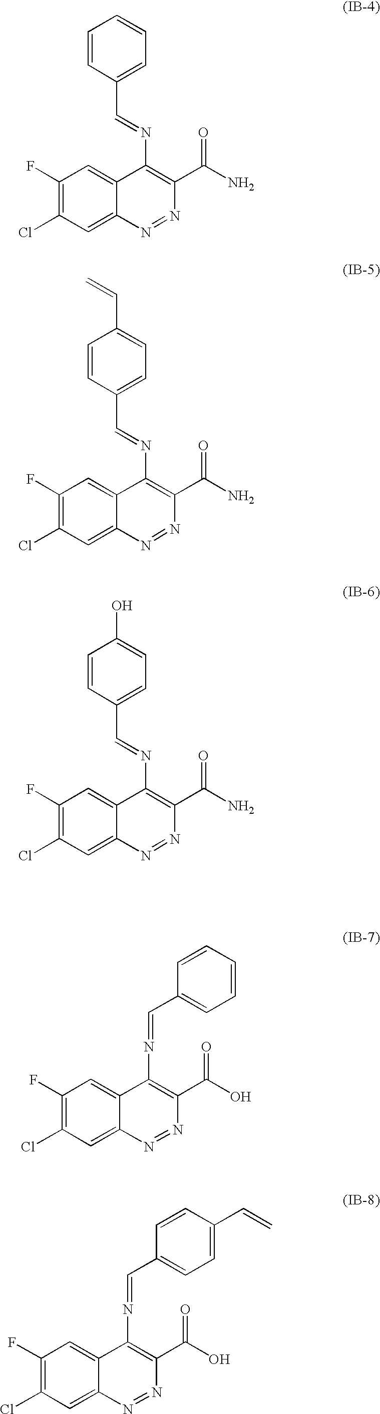

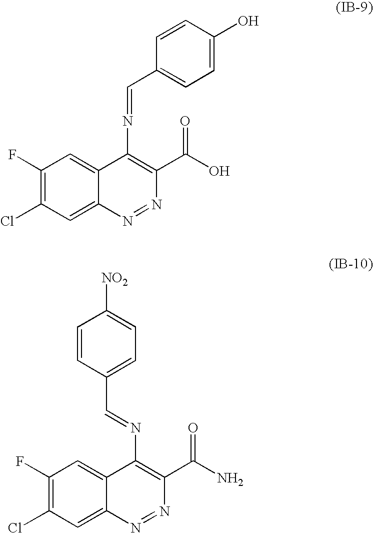

43. The compound of claim 18, where R² and R³ are the same or different and are independently hydrogen, alkoxy, halo, —NO₂, —NH₂, heterocycle or substituted heterocycle.

44. The compound of claim 18, where R² and R³ are the same or different and are independently selected from halo.

45. The compound of claim 18, where R² is —F and R³ is —Cl, and the compound has the following structure (IC-3) below:

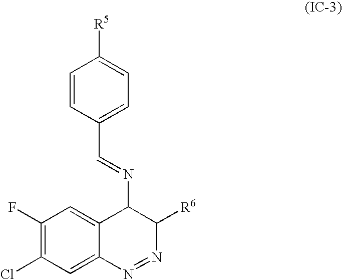

46. The compound of claim 45, where R⁵ is selected from hydrogen, hydroxyl or —C(═C).

47. The compound of claim 45, where R⁶ is selected from —C(═O)—NH2 or —C(═O)OH.

48. The compound of claim 45, where R₅ is selected from hydroxyl or —C(═C) and R₆ is —C(═O)OH.

49. The compound of claim 45, where the compound has a structure selected from the group consisting of structures (IC-4) to (IC-5):

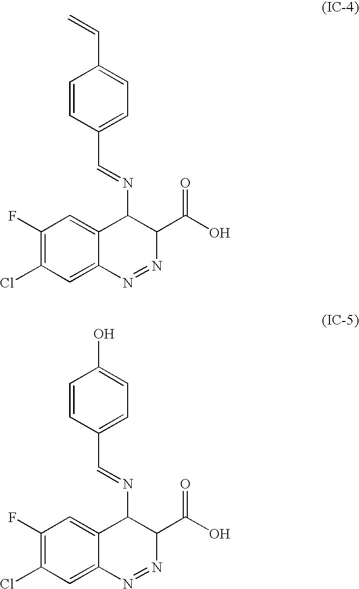

50. A pharmaceutical composition comprising a compound according to claim 1, in combination with a pharmaceutically acceptable carrier.

51. A method for inhibiting protein kinase activity in a biological sample, which method comprises contacting the biological sample with a compound according to claim 1, or a pharmaceutically acceptable composition comprising said compound, and thereby inhibiting protein kinase activity in the sample.

52. A method for treating a protein kinase-mediated disease, which method comprises administering to a subject in need thereof a therapeutically effective amount of a compound according to claim 1, or a pharmaceutically acceptable composition comprising said compound, and thereby treating the protein kinase-mediated disease.

53. The method of claim 52, where the protein kinase-mediated disease is a Plk-1-mediated disease.

54. The method of claim 53, wherein the Plk-1-mediated disease is cancer.

55. The method of claim 54, wherein the cancer is pancreatic cancer, breast cancer, colon cancer, endometrial cancer, esophageal cancer, oropharyngeal cancer, lung cancer or skin cancer.
